(19) United States
(12) Patent Application Publication
(10) Pub. No.: US 2026/0224125 A1
(43) Pub. Date: Aug. 6, 2026

(54) IDENTIFYING EJECTION FRACTION USING A SINGLE LEAD CARDIAC ELECTROGRAM SENSED BY A MEDICAL DEVICE

(71) Applicant: Medtronic, Inc., Minneapolis, MN (US)

(72) Inventors: Neethu Lekshmi Vasudevan Jalaja, Manor, TX (US); Sean R. Landman, Minneapolis, MN (US); Shantanu Sarkar, Roseville, MN (US); Jodi L. Redemske, South Haven, MN (US)

(21) Appl. No.: 19/158,371

(22) PCT Filed: Jan. 5, 2024
(86) PCT No.: PCT/US2024/010436

§ 371 Date: Aug. 20, 2025

Related U.S. Application Data
(60) Provisional application No. 63/487,779, filed on Mar. 1, 2023.

(51) Int. Cl. A61B 5/029; A61B 5/00; A61B 5/318; G16H 50/30

(52) U.S. Cl. CPC A61B 5/029; A61B 5/318; A61B 5/726; A61B 5/7264; G16H 50/30

(57) ABSTRACT

An example system for determining reduced ejection fraction includes two or more electrodes forming a single lead configured to capture a cardiac electrogram (EGM) signal of a patient, circuitry configured to: convert the EGM signal to a time-frequency domain using a continuous wavelet transform; and apply the converted EGM signal to a convolutional neural network to determine one or more of an amount of ejection fraction or a classification of ejection fraction.

This application is an international application with provisional priority of U.S. Provisional Patent Application No. 63/487,779, filed 1 Mar. 2023, the entire content of which is incorporated herein by reference.

### FIELD

The disclosure relates generally to medical device systems and, more particularly, medical device systems configured to determine ejection fraction.

### BACKGROUND

Medical devices may be used to monitor physiological signals of a patient. For example, some medical devices are configured to sense cardiac electrogram (EGM) signals indicative of the electrical activity of the heart via electrodes. Some medical devices may be configured to deliver a therapy in conjunction with or separate from the monitoring of physiological signals.

Ejection fraction (EF) is useful in assessing the overall strength of the heart and left ventricular (LV) systolic performance. However, the standard tool for left ventricular ejection fraction (LVEF) assessment in routine clinical setting, e.g., echocardiography, is limited to use in clinical settings.

### SUMMARY

EGMs may also be used for LV systolic dysfunction diagnosis, e.g., for identification of simple abnormalities on an EGM, or classifying EF using a 12-lead EGM system. In general, this disclosure is directed to techniques for a machine learning model to identify low ejection fraction from a single lead EGM, such as a single lead EGM from an insertable cardiac monitor (ICM) device or other cardiac implantable electronic devices (CIED). A single lead EGM may be continuously sensed and monitored by such devices, e.g., autonomously on a periodic, triggered, or other basis. In this manner, the EGM may be used as a screening tool to identify LV systolic performance without a patient even going to hospital. Such device may be configured to analyze the EGM on board, or to transmit EGMs to other devices for analysis. For example, daily or other periodic EGM transmissions may allow the detection of low LVEF in a cloud platform that may send an alert to a clinician computing device.

In some examples, converting the EGM signal obtained from the single lead to a time-frequency domain using a continuous wavelet transform breaks down various components of the EGM signal. The components may be input into a machine learning model or other artificial intelligence developed algorithm that provides high-frequency resolution and low time resolution at low frequencies and/or provides high time resolution and low-frequency resolution at high frequencies. This may help determine an amount of ejection fraction or a classification of ejection fraction with greater sensitivity and/or specificity than other techniques, or than would otherwise be possible with a single lead EGM.

In some examples in accordance with techniques of this disclosure, processing circuitry of a medical device system may determine ejection fraction, such as reduced ejection fraction, and a clinician may be alerted earlier without a patient needing to go a hospital. Accordingly, medical intervention and/or treatment due to reduced ejection fraction may be applied sooner which may lower the risk of long-term complications of heart failure (HF) patients and may reduce mortality and/or morbidity. In some examples, an alert may be triggered to a clinician to order an actual EF measurement using an echocardiogram or other conventional means to confirm the reduced EF and then determine further therapeutic options.

In addition, since medical device systems described herein may determine an EF of a patient continuously, hourly, and/or daily, dynamic changes in EF may be tracked, which is not possible today, and which will open up new possibilities for treatment recommendations.

In some examples, this disclosure is directed to techniques for determining a HF related hospitalization risk over a future period of time based on an amount of change in HF probability. Processing circuitry of a medical device system may apply single lead EGMs sensed by an IMD for respective time periods to the machine learning model, which may output respective values related to a probability of reduced EF for the time periods and, consequently, related to HF hospitalization for the time periods. The processing circuitry may compare a change in probability over time periods, e.g., between a current time period and a baseline time period, to one or more thresholds, and generate communications or alerts to users and computing devices, or take one or more other actions based on the comparisons, e.g., based on the change in probability exceeding a threshold change indicative of increased risk of HF hospitalization over the future time period.

In some examples in accordance with techniques of this disclosure, a HF related hospitalization risk over a future period of time may be detected before a patient needs to go a hospital. Accordingly, medical intervention and/or treatment due to an increased HF hospitalization risk may be applied sooner which may lower the risk of long-term complications of heart failure patients and may reduce mortality and/or morbidity.

In addition, since the device described herein may determine an EGM(s) of a patient continuously, hourly, and/or daily, dynamic changes in HF probability may be tracked, which is not possible today, and that will open up new possibilities for treatment recommendations.

In an example, a medical system for determining reduced ejection fraction comprises two or more electrodes forming a single lead configured to capture a cardiac electrogram (EGM) signal of a patient; and circuitry configured to: convert the EGM signal to a time-frequency domain using a continuous wavelet transform; and apply the converted EGM signal to a convolutional neural network to determine one or more of an amount of ejection fraction or a classification of ejection fraction.

In an example, a method for operating processing circuity of a medical system comprising receiving, by the processing circuitry, a cardiac electrogram (EGM) signal of a patient obtained by a single lead; converting, by the processing circuitry, the EGM signal to a time-frequency domain using a continuous wavelet transform; and applying, by the processing circuitry, the converted EGM signal to a convolutional neural network to determine one or more of an amount of ejection fraction or a classification of ejection fraction.

In an example, medical system for determining reduced ejection fraction comprising; two or more electrodes forming a single lead configured to capture a baseline cardiac electrogram (EGM) signal of a patient and a follow-up cardiac EGM signal of the patient; and circuitry configured to: determine a baseline heart failure (HF) probability based on the baseline cardiac EGM signal; determine a follow-up HF probability based on the follow-up cardiac EGM signal; determine an amount of change between the baseline HF probability and the follow-up HF probability; compare the determined amount of change to an HF hospitalization threshold; and in response to determining the amount of change between the baseline HF probability and the follow-up HF probability is greater than or equal to the HF hospitalization threshold, output an indication of a risk of hospitalization of the patient over a period of time is high.

The summary is intended to provide an overview of the subject matter described in this disclosure. It is not intended to provide an exclusive or exhaustive explanation of the systems, device, and methods described in detail within the accompanying drawings and description below. Further details of one or more examples of this disclosure are set forth in the accompanying drawings and in the description below. Other features, objects, and advantages will be apparent from the description and drawings, and from the claims.

### BRIEF DESCRIPTION OF THE DRAWINGS

FIG. 1 illustrates the environment of an example medical system in conjunction with a patient, in accordance with one or more techniques disclosed herein.

FIG. 2 is a functional block diagram illustrating an example configuration of the implantable medical device (IMD) of the medical system of FIG. 1, in accordance with one or more techniques disclosed herein.

FIG. 3 is a conceptual side-view diagram illustrating an example configuration of the IMD of FIGS. 1 and 2, in accordance with one or more techniques disclosed herein.

FIG. 4 is a functional block diagram illustrating an example configuration of the external device of FIG. 1, in accordance with one or more techniques disclosed herein.

FIG. 5 is a block diagram illustrating an example system that includes an access point, a network, external computing devices, such as a server, and one or more other computing devices, which may be coupled to the IMD and external device of FIGS. 1-4, in accordance with one or more techniques disclosed herein.

FIG. 6A illustrates an example of decomposing an EGM signal from a one-dimensional lead into time-frequency components, in accordance with one or more techniques disclosed herein.

FIG. 6B illustrates an example of converting an EGM signal from a one-dimensional lead into a corresponding time-frequency representation and converting the time-frequency representation of the EGM signal from a one-dimensional lead into a scalogram, in accordance with one or more techniques disclosed herein.

FIG. 7 illustrates examples of wavelet transform to differentiate the time-frequency resolution, in accordance with one or more techniques disclosed herein.

FIG. 8A illustrates an example of applying an EGM signal from a one-dimensional lead to a convolutional neural network, in accordance with one or more techniques disclosed herein.

FIG. 8B illustrates an example of converting an EGM signal from a one-dimensional lead to a two-dimensional array and applying the two-dimensional array to a convolutional neural network, in accordance with one or more techniques disclosed herein.

FIG. 9A is a chart illustrating an example of a confusion matrix, in accordance with one or more techniques disclosed herein.

FIG. 9B is a graph illustrating an example of a receiver operating characteristic (ROC) curve, in accordance with one or more techniques disclosed herein.

FIG. 10 is a graph illustrating example Kaplan Meier curves of HF hospitalization event estimates,

FIGS. 11A and 11B are conceptual diagrams illustrating example training processes for an artificial intelligence model, in accordance with examples of the current disclosure.

FIG. 12 is a conceptual diagram illustrating an example machine learning model configured to determine the amount of ejection fraction or a classification of ejection fraction.

FIG. 13 is a flow diagram illustrating an example technique for operating a system to determine an amount of ejection fraction or a classification of ejection fraction.

FIG. 14 is a flow diagram illustrating an example technique for operating a system to output an indication of a risk of hospitalization of the patient over a period of time is high.

Like reference characters denote like elements throughout the description and figures.

### DETAILED DESCRIPTION

A variety of types of medical devices sense cardiac EGMs. Some medical devices that sense cardiac EGMs are non-invasive, e.g., using a plurality of electrodes placed in contact with external portions of the patient, such as at various locations on the skin of the patient. The electrodes used to monitor the cardiac EGM in these non-invasive processes may be attached to the patient using an adhesive, strap, belt, or vest, as examples, and electrically coupled to a monitoring device, such as an electrocardiograph, Holter monitor, or other electronic device. The electrodes are configured to sense electrical signals associated with the electrical activity of the heart or other cardiac tissue of the patient, and to provide these sensed electrical signals to the electronic device for further processing and/or display of the electrical signals. The non-invasive devices and methods may be utilized on a temporary basis, for example to monitor a patient during a clinical visit, such as during a doctor's appointment, or for example for a predetermined period of time, for example for one day (twenty-four hours), or for a period of several days.

External devices that may be used to non-invasively sense and monitor cardiac EGMs include wearable devices with electrodes configured to contact the skin of the patient, such as patches, watches, or necklaces. One example of a wearable physiological monitor configured to sense a cardiac EGM is the SEEQ™ Mobile Cardiac Telemetry System, which was available from Medtronic, Inc., of Minneapolis, Minnesota. Such external devices may facilitate relatively longer-term monitoring of patients during normal daily activities, and may periodically transmit collected data to a network service, such as the Medtronic Carelink™ Network.

Some implantable medical devices (IMDs) also sense and monitor cardiac EGMs. The electrodes used by IMDs to sense cardiac EGMs are typically integrated with a housing of the IMD and/or coupled to the IMD via one or more elongated leads. Example IMDs that monitor cardiac EGMs include pacemakers and implantable cardioverter-defibrillators, which may be coupled to intravascular or extravascular leads, as well as pacemakers with housings configured for implantation within the heart, which may be leadless. An example of pacemaker configured for intracardiac implantation is the Micra™ Transcatheter Pacing System, available from Medtronic, Inc. Some IMDs that do not provide therapy, e.g., implantable patient monitors, sense cardiac EGMs. Examples of such an IMD are the Reveal LINQ™ and LINQ II™ Insertable Cardiac Monitors, available from Medtronic, Inc., which may be inserted subcutaneously. Such IMDs may facilitate relatively longer-term monitoring of patients during normal daily activities and may periodically transmit collected data to a network service, such as the Medtronic Carelink™ Network.

Any medical device configured to sense a cardiac EGM via implanted or external electrodes, including the examples identified herein, may implement the techniques of this disclosure for evaluating a cardiac EGM to determine an amount of ejection fraction of a patient. The techniques herein include determining ejection fraction from an EGM obtained from a single lead, such as from an IMD. The EGM may be used as a screening tool to identify LV systolic performance without a patient even going to hospital. Daily EGM transmission may allow the detection of low LVEF in a cloud platform that may send an alert to a clinician computing device.

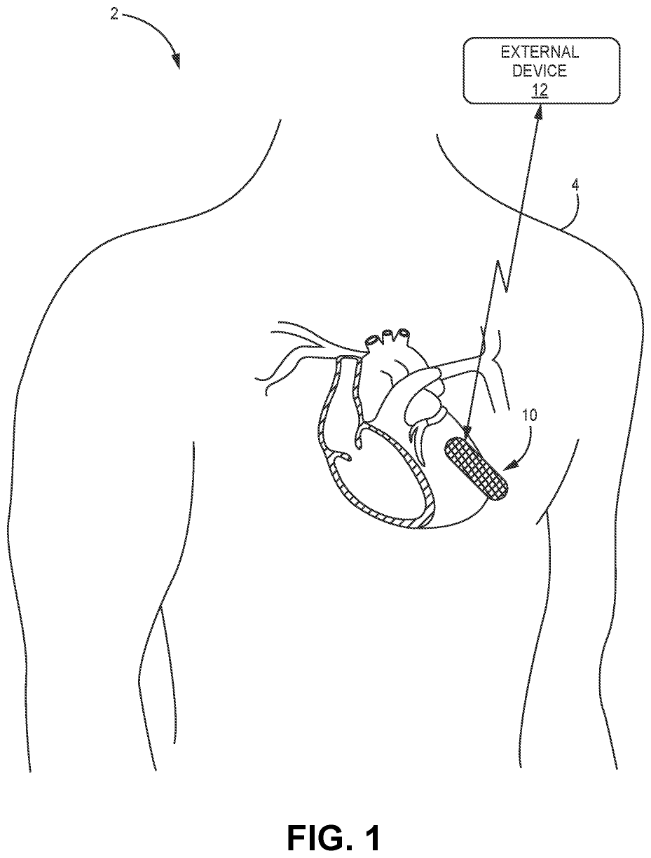

FIG. 1 illustrates the environment of an example medical system 2 in conjunction with a patient 4, in accordance with one or more techniques of this disclosure. The example techniques may be used with an IMD 10, which may be in wireless communication with at least one of external device 12 and other devices not pictured in FIG. 1. In some examples, IMD 10 is implanted outside of a thoracic cavity of patient 4 (e.g., subcutaneously in the pectoral location illustrated in FIG. 1). IMD 10 may be positioned near the sternum near or just below the level of the heart of patient 4, e.g., at least partially within the cardiac silhouette. IMD 10 includes a plurality of electrodes (not shown in FIG. 1) and is configured to sense a cardiac EGM via the plurality of electrodes. In some examples, IMD 10 takes the form of the Reveal LINQ™ or LINQ II™ ICM, or another ICM similar to, e.g., a version or modification of, the Reveal LINQ™ or LINQ II™ ICMS.

External device 12 may be a computing device with a display viewable by the user and an interface for providing input to external device 12 (i.e., a user input mechanism). In some examples, external device 12 may be a notebook computer, tablet computer, workstation, one or more servers, cellular phone, personal digital assistant, or another computing device that may run an application that enables the computing device to interact with IMD 10. External device 12 is configured to communicate with IMD 10 and, optionally, another computing device (not illustrated in FIG. 1), via wireless communication. External device 12, for example, may communicate via near-field communication technologies (e.g., inductive coupling, NFC or other communication technologies operable at ranges less than 10-20 cm) and far-field communication technologies (e.g., RF telemetry according to the 802.11 or Bluetooth ® specification sets, or other communication technologies operable at ranges greater than near-field communication technologies).

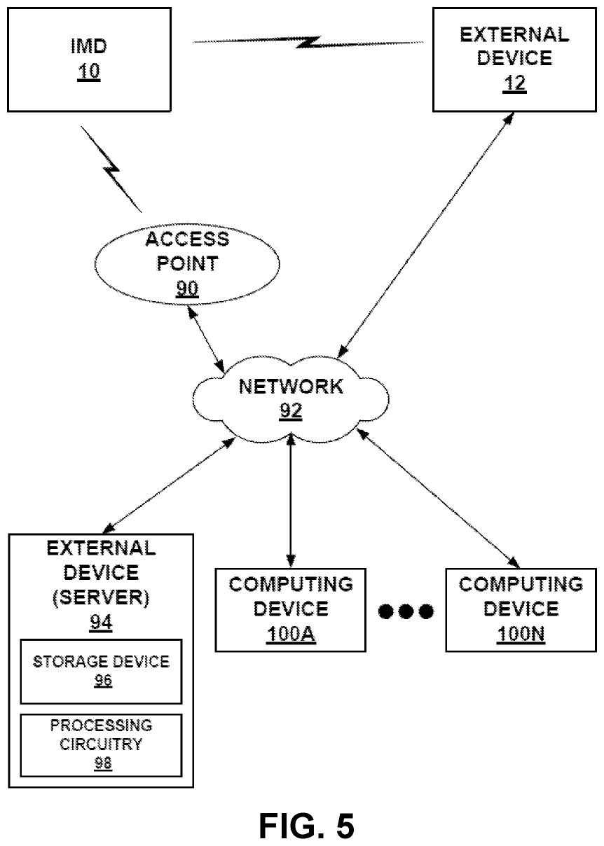

External device 12 may be used to configure operational parameters for IMD 10. External device 12 may be used to retrieve data from IMD 10. The retrieved data may include values of physiological parameters measured by IMD 10, indications of episodes of arrhythmia or other maladies detected by IMD 10, and physiological signals recorded by IMD 10. For example, external device 12 may retrieve cardiac EGM segments recorded by IMD 10. As discussed in greater detail below with respect to FIG. 5, one or more remote computing devices may interact with IMD 10 in a manner similar to external device 12, e.g., to program IMD 10 and/or retrieve data from IMD 10, via a network.

Processing circuitry of medical system 2, e.g., of IMD 10, external device 12, and/or of one or more other computing devices, may be configured to perform the example techniques of this disclosure for determining an amount of ejection fraction. In some examples, the processing circuitry of medical system 2 analyzes a cardiac EGM sensed by IMD 10 to determine an amount of ejection fraction based on converting the cardiac EGM signal to a time-frequency domain using a continuous wavelet transform and applying the converted EGM signal to a convolutional neural network to determine an amount of ejection fraction. In some examples, processing circuitry may determine a raw number of the amount of ejection fraction using a regression model or it may classify the amount of ejection fraction into subsets, such as low, medium, or high. Although described in the context of examples in which IMD 10 that senses the cardiac EGM comprises an insertable cardiac monitor, example systems including one or more implantable or external devices of any type configured to sense a cardiac EGM may be configured to implement the techniques of this disclosure.

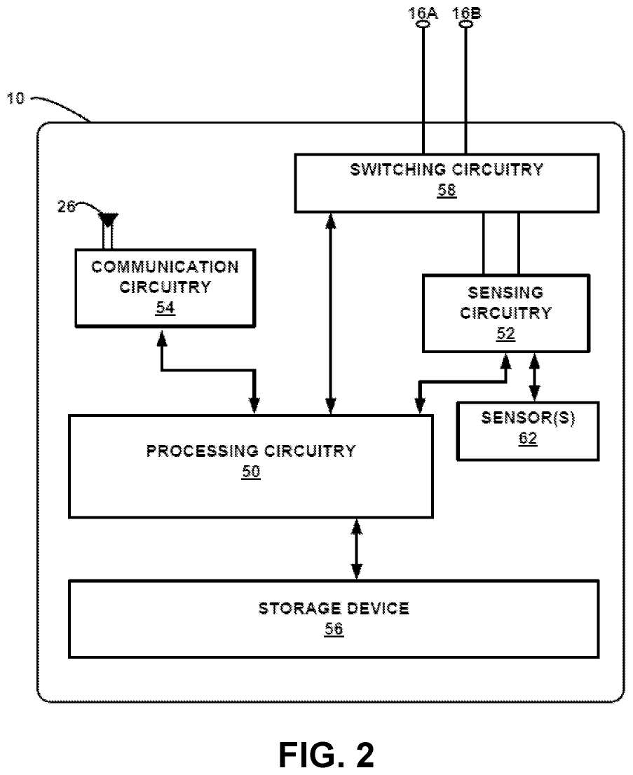

FIG. 2 is a functional block diagram illustrating an example configuration of IMD 10 of FIG. 1 in accordance with one or more techniques described herein. In the illustrated example, IMD 10 includes electrodes 16A and 16B (collectively “electrodes 16”), antenna 26, processing circuitry 50, sensing circuitry 52, communication circuitry 54, storage device 56, switching circuitry 58, and sensors 62. Although the illustrated example includes two electrodes 16, IMDs including or coupled to more than two electrodes 16 may implement the techniques of this disclosure in some examples.

Processing circuitry 50 may include fixed function circuitry and/or programmable processing circuitry. Processing circuitry 50 may include any one or more of a microprocessor, a controller, a digital signal processor (DSP), an application specific integrated circuit (ASIC), a field-programmable gate array (FPGA), or equivalent discrete or analog logic circuitry. In some examples, processing circuitry 50 may include multiple components, such as any combination of one or more microprocessors, one or more controllers, one or more DSPs, one or more ASICs, or one or more FPGAs, as well as other discrete or integrated logic circuitry. The functions attributed to processing circuitry 50 herein may be embodied as software, firmware, hardware or any combination thereof.

Sensing circuitry 52 may be selectively coupled to electrodes 16 via switching circuitry 58, e.g., to select the electrodes 16 and polarity, referred to as the sensing vector, used to sense a cardiac EGM, as controlled by processing circuitry 50. Sensing circuitry 52 may sense signals from electrodes 16, e.g., to produce a cardiac EGM, in order to facilitate monitoring the electrical activity of the heart. Sensing circuitry 52 also may monitor signals from sensors 62, which may include one or more accelerometers, pressure sensors, and/or optical sensors, as examples. In some examples, sensing circuitry 52 may include one or more filters and amplifiers for filtering and amplifying signals received from electrodes 16 and/or sensors 62.

Sensing circuitry 52 and/or processing circuitry 50 may be configured to detect cardiac depolarizations (e.g., P-waves of atrial depolarizations or R-waves of ventricular depolarizations) when the cardiac EGM amplitude crosses a sensing threshold. For cardiac depolarization detection, sensing circuitry 52 may include a rectifier, filter, amplifier, comparator, and/or analog-to-digital converter, in some examples. In some examples, sensing circuitry 52 may output an indication to processing circuitry 50 in response to sensing of a cardiac depolarization. In this manner, processing circuitry 50 may receive detected cardiac depolarization indicators corresponding to the occurrence of detected R-waves and P-waves in the respective chambers of heart. Processing circuitry 50 may use the indications of detected R-waves and P-waves for determining inter-depolarization intervals, heart rate, and detecting arrhythmias, such as tachyarrhythmias and asystole. In some examples, the cardiac EGM should be sensed during normal sinus rhythm to determine ejection fraction. In particular, cardiac EGMs with one or more of a premature ventricular contraction (PVC), ventricular fibrillation (VF), ventricular tachycardia (VT), or other ventricular arrhythmias should be avoided when determining an amount of ejection fraction. Accordingly, processing circuitry 50 may determine whether a cardiac EGM includes one or more of PVC, VF, VT, or other ventricular arrhythmias and determine not to use the cardiac EGM to determine ejection fraction in response to determining the cardiac EGM includes one or more of PVC, VF, VT, or other ventricular arrhythmias.

Sensing circuitry 52 may also provide one or more digitized cardiac EGM signals to processing circuitry 50 for analysis, e.g., for use in cardiac rhythm discrimination, and/or for analysis to determine an amount of ejection fraction according to the techniques of this disclosure. In some examples, processing circuitry 50 may store the digitized cardiac EGM in storage device 56. Processing circuitry 50 of IMD 10, and/or processing circuitry of another device that retrieves data from IMD 10, may analyze the cardiac EGM to determine an amount of ejection fraction according to the techniques of this disclosure.

Communication circuitry 54 may include any suitable hardware, firmware, software or any combination thereof for communicating with another device, such as external device 12, another networked computing device, or another IMD or sensor. Under the control of processing circuitry 50, communication circuitry 54 may receive downlink telemetry from, as well as send uplink telemetry to external device 12 or another device with the aid of an internal or external antenna, e.g., antenna 26. In addition, processing circuitry 50 may communicate with a networked computing device via an external device (e.g., external device 12) and a computer network, such as the Medtronic CareLink® Network. Antenna 26 and communication circuitry 54 may be configured to transmit and/or receive signals via inductive coupling, electromagnetic coupling, Near Field Communication (NFC), Radio Frequency (RF) communication, Bluetooth, WiFi, or other proprietary or non-proprietary wireless communication schemes.

In some examples, storage device 56 includes computer-readable instructions that, when executed by processing circuitry 50, cause IMD 10 and processing circuitry 50 to perform various functions attributed to IMD 10 and processing circuitry 50 herein. Storage device 56 may include any volatile, non-volatile, magnetic, optical, or electrical media, such as a random access memory (RAM), read-only memory (ROM), non-volatile RAM (NVRAM), electrically-erasable programmable ROM (EEPROM), flash memory, or any other digital media. Storage device 56 may store, as examples, programmed values for one or more operational parameters of IMD 10 and/or data collected by IMD 10 for transmission to another device using communication circuitry 54. Data stored by storage device 56 and transmitted by communication circuitry 54 to one or more other devices may include digitized cardiac EGMs, as examples.

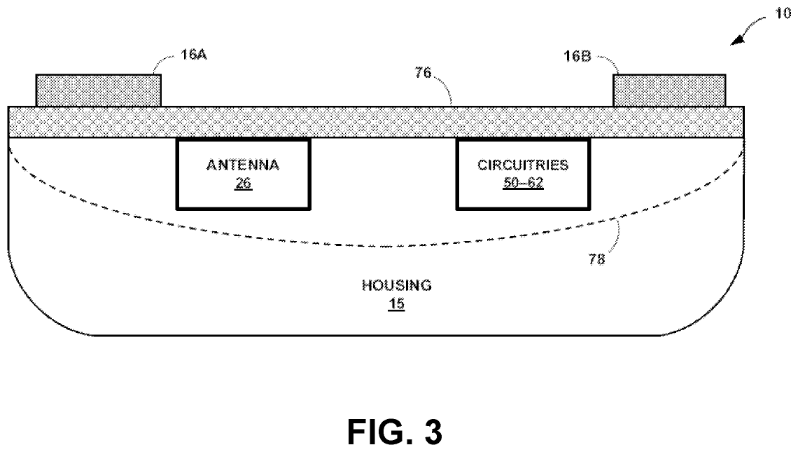

FIG. 3 is a conceptual side-view diagram illustrating an example configuration of IMD 10 of FIGS. 1 and 2. In the example shown in FIG. 3, IMD 10 may include a leadless, subcutaneously-implantable monitoring device having a housing 15 and an insulative cover 76. Electrode 16A and electrode 16B may be formed or placed on an outer surface of cover 76. Circuitries 50-62, described above with respect to FIG. 2, may be formed or placed on an inner surface of cover 76, or within housing 15. In the illustrated example, antenna 26 is formed or placed on the inner surface of cover 76, but may be formed or placed on the outer surface in some examples. In some examples, one or more of sensors 62 may be formed or placed on the outer surface of cover 76. In some examples, insulative cover 76 may be positioned over an open housing 15 such that housing 15 and cover 76 enclose antenna 26 and circuitries 50-62, and protect the antenna and circuitries from fluids such as body fluids.

One or more of antenna 26 or circuitries 50-62 may be formed on the inner side of insulative cover 76, such as by using flip-chip technology. Insulative cover 76 may be flipped onto a housing 15. When flipped and placed onto housing 15, the components of IMD 10 formed on the inner side of insulative cover 76 may be positioned in a gap 78 defined by housing 15, Electrodes 16 may be electrically connected to switching circuitry 58 through one or more vias (not shown) formed through insulative cover 76. Insulative cover 76 may be formed of sapphire (i.e., corundum), glass, parylene, and/or any other suitable insulating material. Housing 15 may be formed from titanium or any other suitable material (e.g., a biocompatible material). Electrodes 16 may be formed from any of stainless steel, titanium, platinum, iridium, or alloys thereof. In addition, electrodes 16 may be coated with a material such as titanium nitride or fractal titanium nitride, although other suitable materials and coatings for such electrodes may be used.

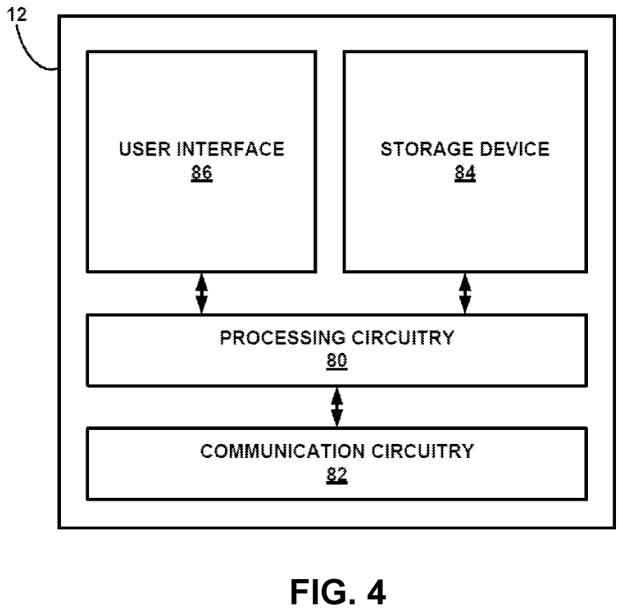

FIG. 4 is a block diagram illustrating an example configuration of components of external device 12. In the example of FIG. 4, external device 12 includes processing circuitry 80, communication circuitry 82, storage device 84, and user interface 86.

Processing circuitry 80 may include one or more processors that are configured to implement functionality and/or process instructions for execution within external device 12. For example, processing circuitry 80 may be capable of processing instructions stored in storage device 84. Processing circuitry 80 may include, for example, microprocessors, DSPs, ASICs, FPGAs, or equivalent discrete or integrated logic circuitry, or a combination of any of the foregoing devices or circuitry. Accordingly, processing circuitry 80 may include any suitable structure, whether in hardware, software, firmware, or any combination thereof, to perform the functions ascribed herein to processing circuitry 80.

Communication circuitry 82 may include any suitable hardware, firmware, software or any combination thereof for communicating with another device, such as IMD 10. Under the control of processing circuitry 80, communication circuitry 82 may receive downlink telemetry from, as well as send uplink telemetry to, IMD 10, or another device. Communication circuitry 82 may be configured to transmit or receive signals via inductive coupling, electromagnetic coupling, Near Field Communication (NFC), Radio Frequency (RF) communication, Bluetooth, WiFi, or other proprietary or non-proprietary wireless communication schemes. Communication circuitry 82 may also be configured to communicate with devices other than IMD 10 via any of a variety of forms of wired and/or wireless communication and/or network protocols.

Storage device 84 may be configured to store information within external device 12 during operation. Storage device 84 may include a computer-readable storage medium or computer-readable storage device. In some examples, storage device 84 includes one or more of a short-term memory or a long-term memory. Storage device 84 may include, for example, RAM, DRAM, SRAM, magnetic discs, optical discs, flash memories, or forms of EPROM or EEPROM. In some examples, storage device 84 is used to store data indicative of instructions for execution by processing circuitry 80. Storage device 84 may be used by software or applications running on external device 12 to temporarily store information during program execution.

Data exchanged between external device 12 and IMD 10 may include operational parameters. External device 12 may transmit data including computer readable instructions which, when implemented by IMD 10, may control IMD 10 to change one or more operational parameters and/or export collected data. For example, processing circuitry 80 may transmit an instruction to IMD 10 which requests IMD 10 to export collected data (e.g., digitized cardiac EGMs) to external device 12. In tum, external device 12 may receive the collected data from IMD 10 and store the collected data in storage device 84. Processing circuitry 80 may implement any of the techniques described herein to analyze cardiac EGMs received from IMD 10, e.g., to determine an amount of ejection fraction.

A user, such as a clinician or patient 4, may interact with external device 12 through user interface 86. User interface 86 includes a display (not shown), such as a liquid crystal display (LCD) or a light emitting diode (LED) display or other type of screen, with which processing circuitry 80 may present information related to IMD 10, e.g., cardiac EGMs. In addition, user interface 86 may include an input mechanism to receive input from the user. The input mechanisms may include, for example, any one or more of buttons, a keypad (e.g., an alphanumeric keypad), a peripheral pointing device, a touch screen, or another input mechanism that allows the user to navigate through user interfaces presented by processing circuitry 80 of external device 12 and provide input. In other examples, user interface 86 also includes audio circuitry for providing audible notifications, instructions or other sounds to the user, receiving voice commands from the user, or both.

FIG. 5 is a block diagram illustrating an example system that includes an access point 90, a network 92, external computing devices, such as a server 94, and one or more other computing devices 100A-100N (collectively, “computing devices 100”), which may be coupled to IMD 10 and external device 12 via network 92, in accordance with one or more techniques described herein. In this example, IMD 10 may use communication circuitry 54 to communicate with external device 12 via a first wireless connection, and to communicate with an access point 90 via a second wireless connection. In the example of FIG. 5, access point 90, external device 12, server 94, and computing devices 100 are interconnected and may communicate with each other through network 92.

Access point 90 may include a device that connects to network 92 via any of a variety of connections, such as telephone dial-up, digital subscriber line (DSL), or cable modem connections. In other examples, access point 90 may be coupled to network 92 through different forms of connections, including wired or wireless connections. In some examples, access point 90 may be a user device, such as a tablet or smartphone, that may be co-located with the patient. IMD 10 may be configured to transmit data, such as cardiac EGMs, to access point 90. Access point 90 may then communicate the retrieved data to server 94 via network 92.

In some cases, server 94 may be configured to provide a secure storage site for data that has been collected from IMD 10 and/or external device 12. In some cases, server 94 may assemble data in web pages or other documents for viewing by trained professionals, such as clinicians, via computing devices 100. One or more aspects of the illustrated system of FIG. 5 may be implemented with general network technology and functionality, which may be similar to that provided by the Medtronic CareLink(R) Network.

In some examples, one or more of computing devices 100 may be a tablet or other smart device located with a clinician, by which the clinician may program, receive alerts from, and/or interrogate IMD 10. For example, the clinician may access data collected by IMD 10 through a computing device 100, such as when patient 4 is in in between clinician visits, to check on a status of a medical condition. In some examples, the clinician may enter instructions for a medical intervention for patient 4 into an application executed by computing device 100, such as based on a status of a patient condition determined by IMD 10, external device 12, server 94. or any combination thereof, or based on other patient data known to the clinician. Device 100 then may transmit the instructions for medical intervention to another of computing devices 100 located with patient 4 or a caregiver of patient 4. For example, such instructions for medical intervention may include an instruction to change a drug dosage, timing, or selection, to schedule a visit with the clinician, or to seek medical attention. In further examples, a computing device 100 may generate an alert to patient 4 based on a status of a medical condition of patient 4, which may enable patient 4 proactively to seek medical attention prior to receiving instructions for a medical intervention. In this manner, patient 4 may be empowered to take action, as needed, to address his or her medical status, which may help improve clinical outcomes for patient 4.

In the example illustrated by FIG. 5, server 94 includes a storage device 96, e.g., to store data retrieved from IMD 10, and processing circuitry 98. Although not illustrated in FIG. S computing devices 100 may similarly include a storage device and processing circuitry. Processing circuitry 98 may include one or more processors that are configured to implement functionality and/or process instructions for execution within server 94. For example, processing circuitry 98 may be capable of processing instructions stored in memory 96. Processing circuitry 98 may include, for example, microprocessors, DSPs, ASICs, FPGAs, or equivalent discrete or integrated logic circuitry, or a combination of any of the foregoing devices or circuitry. Accordingly, processing circuitry 98 may include any suitable structure, whether in hardware, software, firmware, or any combination thereof, to perform the functions ascribed herein to processing circuitry 98. Processing circuitry 98 of server 94 and/or the processing circuity of computing devices 100 may implement any of the techniques described herein to analyze cardiac EGMs received from IMD 10.

Storage device 96 may include a computer-readable storage medium or computer-readable storage device. In some examples, memory 96 includes one or more of a short-term memory or a long-term memory. Storage device 96 may include, for example, RAM, DRAM, SRAM, magnetic discs, optical discs, flash memories, or forms of EPROM or EEPROM. In some examples, storage device 96 is used to store data indicative of instructions for execution by processing circuitry 98.

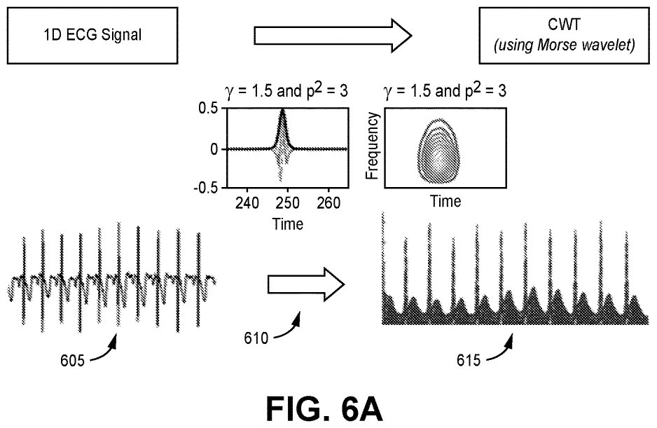

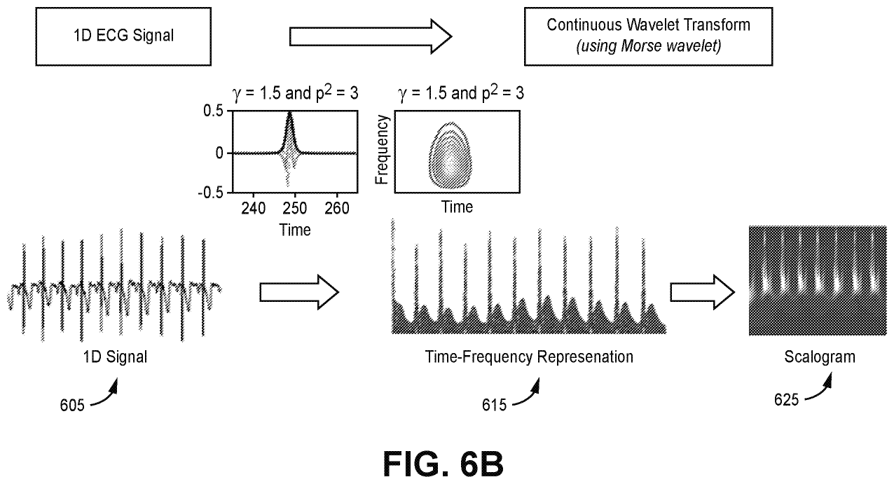

Although the techniques for determining one or more of an amount of ejection fraction or a classification of ejection fraction are described herein primarily (e.g., with respect to FIGS. 6A-10) as being performed by processing circuitry 50 of IMD 10, such techniques may be performed, in whole or part, by processing circuitry of any one or more devices of system 2, such as processing circuitry 80 of external device 12, processing circuitry 98 of server 94, or processing circuitry of one or more computing devices 100.

Ejection fraction (EF) is useful in assessing the overall strength of the heart and left ventricular (LV) systolic performance. The techniques described herein provide a deep learning method to identify low ejection fraction single lead EGM, such as from IMD 10. In some examples, IMD 10 may be an insertable cardiac monitor (ICM) or other CIED (cardiac implantable electronic device) with EGM transmission capability. The EGM may be used as a screening tool to identify LV systolic performance without the patient 4 even going to hospital. In some examples, daily EGM transmission may allow the detection of low LVEF in a cloud platform that may send an alert to a clinician computing device.

In some examples, IMD 10 may obtain EGM data from a single lead EGM routinely, such as, but not limited or, hourly, once every 12 hours, daily, nightly, weekly, etc. In some examples, IMD 10 may determine LVEF based on corresponding EGM data routinely, such as, but not limited or, hourly, daily, nightly, weekly, bi-weekly, etc. Other devices, such as external device 12, server 94, and computing devices 110, may similarly determine LVEF based on transmissions of sensed EGMs from IMD 10, e.g., a digitized segment of a number of minutes of EGM each day. In some examples, IMD 10 may transmit obtained EGM data and/or corresponding LVEF to external device 12. In some examples, IMD 10 may determine LVEF within a period of time of obtaining EGM data, such as, but not limited to, 1 week, 2 weeks, 1 month, etc. In some examples, EGM data may be obtained from an electronic health record (EHR) dataset.

In some examples, LVEF data may be compared to an LVEF threshold to determine whether the LVEF data is categorized as heart failure or normal. For example, the LVEF data may be categorized as heart failure if LVEF is less than or equal to an LVEF threshold. The LVEF data may be categorized as normal is LVEF is greater than the threshold. In some examples, the LVEF threshold may be 35%. The LVEF threshold may be set at a different amount. In this example, if the LVEF is less than or equal to 35%, the LVEF is classified as heart failure. If the LVEF is greater than 35%, the LVEF is classified as normal. In some examples, the LVEF threshold may be variable based on various physiological parameters from patient 4 that may include patient medical history and/or demographic and other information of patient 4, such as age, gender, race, height, weight, and body mass index (BMI).

In some examples, processing circuitry 50 may determine an amount of change in ejection fraction over a period of time and determine heart failure risk of the patient 4 based on the change in ejection fraction over the period of time. For example, processing circuitry 50 may determine whether ejection fraction of patient 12 dropped a certain amount over a period of time, such as ejection fraction dropping more than 20% (e.g., 60% to 39%) over a week, two weeks, month etc. In an example where processing circuitry 50 determines ejection fraction decreases from 60% to 39% over a period of time, while processing circuitry 50 may determine the ejection fraction amount may be greater than a LVEF threshold, as discussed in the example above, processing circuitry 50 may determine an increased risk of heart failure based on the amount of reduction of ejection fraction over the period of time.

In some examples, processing circuitry 50 may convert the EGM signal obtained from the single lead to a time-frequency domain using a continuous wavelet transform to break down various components of the EGM signal to be input into a machine learning model or other artificial intelligence developed algorithm. For example, processing circuitry 50 may use Morse wavelet to perform the wavelet transform. In some examples, processing circuitry 50 may use one or more of principal components, independent components, variational auto encoders, etc. as either an alternative to or in addition to the continuous wavelet transform to break down various components of the EGM signal to be input into a machine learning model or other artificial intelligence developed algorithm. As shown as an example in FIG. 6A, processing circuitry 50 may select relevant wavelet components from the converted wavelet transform and may store the selected relevant wavelet components in a 2D array. In some examples, the 2D array may be used, such as by processing circuitry 50, as input for a machine learning model or other developed algorithms. For example, processing circuitry 50 may convert, such as with a wavelet transform, an EGM signal 605 from a one-dimensional lead into a corresponding time-frequency representation 615, such as a two-dimensional array or sequence.

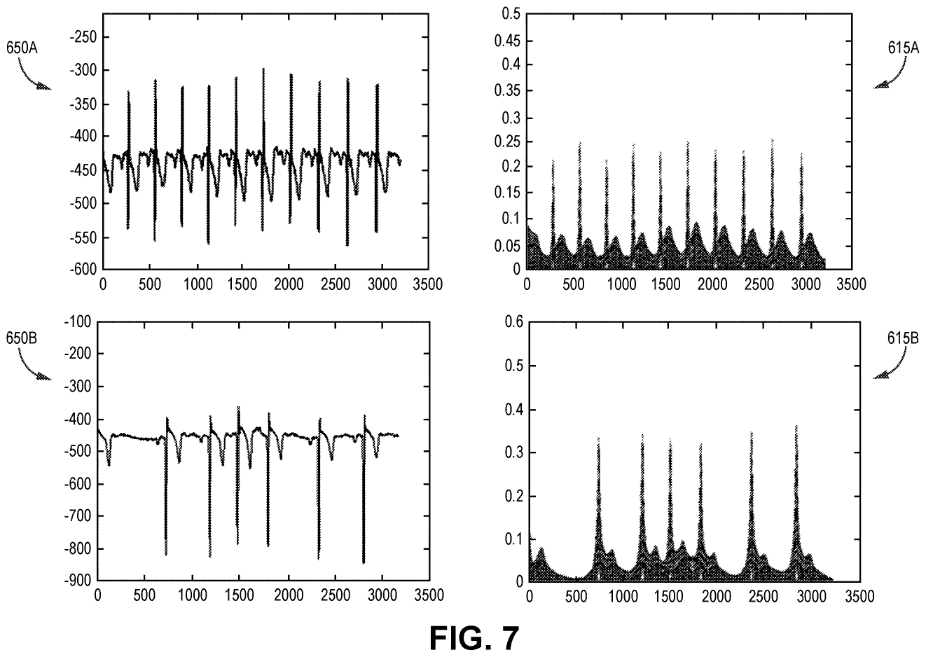

As shown as an example in FIG. 6B, processing circuitry 50 may convert, such as with a wavelet transform, an EGM signal 605 from a one-dimensional lead into a corresponding time-frequency representation 615, such as a two-dimensional array or sequence. In some examples, processing circuitry 50 may convert the time-frequency representation 615 of the EGM signal into image(s), such as scalogram(s) 625. In some examples, the image(s), such as a scalogram 625, may be used as input for a machine learning model or other artificial intelligence developed algorithm. As shown as an example in FIG. 7, a wavelet transform may convert an EGM signal 605A from a one-dimensional lead into a corresponding time-frequency representation 615A to provide high-frequency resolution and low time resolution at low frequencies. In addition, a wavelet transform may also convert an EGM signal 605B from a one-dimensional lead into a corresponding time-frequency representation 615B to provide high time resolution and low-frequency resolution at high frequencies.

In some examples, processing circuitry 50 may transform the EGM signal to enhance features like QRS width, R-wave slews, etc. that are modified because of changes in ejection fraction and feed those into one or more networks, such as a convolutional neural network (CNN), U-nets, recurrent neural nets, etc. Additionally, features may be extracted which are related to changes in these EGM characteristics and can be fed into the networks. In addition, further additional measurements, such as measurements sensed by IMD 10, may also be used as input to predict and/or determine ejection fraction, such as high-resolution accelerometer measurements for heart sounds, high resolution impedance measurements for measurement of fluid and respiration, heart rate and heart rate dynamics (like HRV), tissue oxygenation/perfusion measured by optical sensor, high resolution temperature changes in periphery, etc.

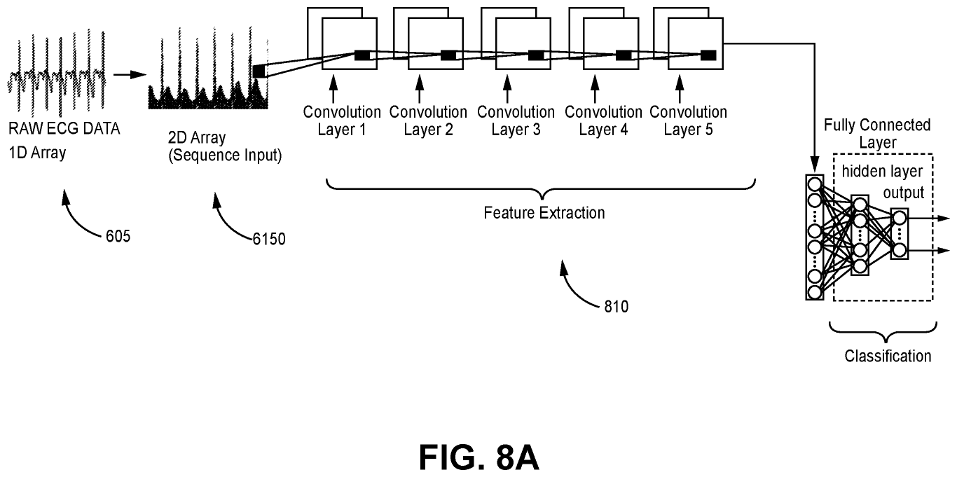

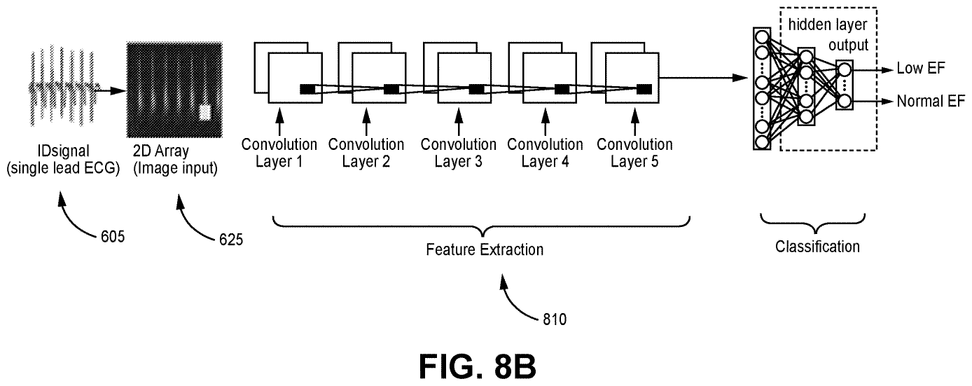

As shown as an example in FIG. 8A, processing circuitry 50 may apply the converted wavelet transform into a one-dimensional CNN model 810 to determine an amount of ejection fraction. In some examples, processing circuitry 50 may convert an EGM signal 605 from a one-dimensional lead into a corresponding two-dimensional array or sequence of time-frequency representation 615 of the EGM signal and apply the two-dimensional array or sequence of time-frequency representation 615 of the EGM signal into a one-dimensional CNN model 810 to determine an amount of ejection fraction The convolutional layers and pooling layers in the CNN model may extract features from the converted wavelet transform, followed by fully connected layers for final classification of LVEF category, such as heart failure or normal. Processing circuitry 50 may cause the determined/classified LVEF category and/or the determined amount of ejection fraction be output to an external computing device, such as a clinician's computing device, to determine treatments to be delivered and/or recommend additional testing based on the determined amount of ejection fraction and/or the determined/classified LVEF category.

As shown as an example in FIG. 8B, processing circuitry 50 may apply the scalogram(s) 625 into a one-dimensional CNN model 810 to determine an amount of ejection fraction. The convolutional layers and pooling layers in the CNN model may extract features from the scalogram(s) 625, followed by fully connected layers for final classification of LVEF category, such as heart failure or normal. Processing circuitry 50 may cause the determined/classified LVEF category and/or the determined amount of ejection fraction be output to an external computing device, such as a clinician's computing device, to determine treatments to be delivered and/or recommend additional testing based on the determined amount of ejection fraction and/or the determined/classified LVEF category.

In accordance with techniques of this disclosure, reduced ejection fraction may be detected earlier without a patient needing to go a hospital. Accordingly, medical intervention and/or treatment due to reduced ejection fraction may be applied sooner which may lower the risk of long-term complications of heart failure patients and may reduce mortality and/or morbidity.

In some examples, in training of CNN model 810, such as shown CNN model 810 in FIGS. 8A and 8B, cross entropy may be used for calculation of loss function and an adaptive moment estimation may be used as optimizer. As an example, the initial learning rate may be selected as 0.0001, epochs as 20 and mini batch size as 64.

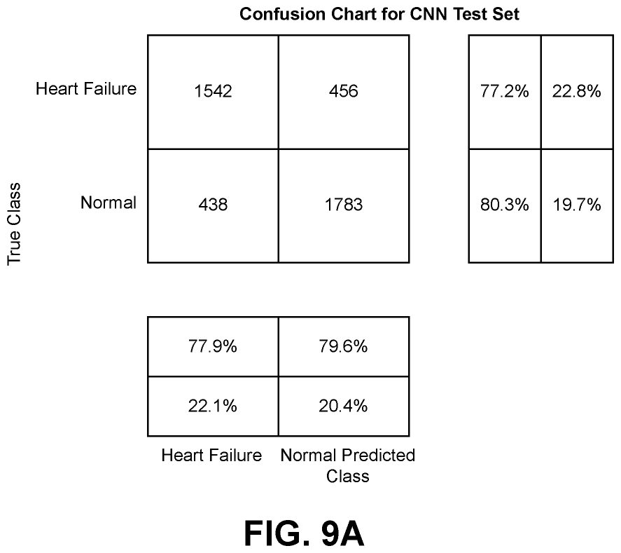

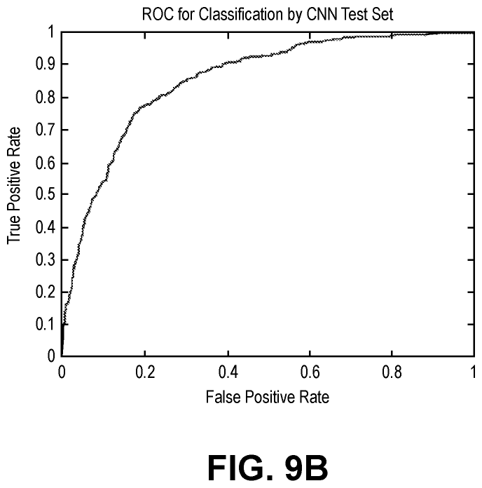

In some examples, to evaluate the performance of the model, six different metrics were be considered: Accuracy, Sensitivity, Specificity, area under curve (AUC), precision and F1 score. FIG. 9A is a chart illustrating an example of a confusion matrix and FIG. 9B is a graph illustrating an example of a receiver operating characteristic (ROC) curve. FIGS. 9A, 9B show illustrate an example where the AUC is 0.85 with sensitivity of 77% and specificity of 80%.

Although the techniques below are described as being performed by processing circuitry 50 of IMD 10, such techniques may be performed, in whole or part, by processing circuitry of any one or more devices of system 2, such as processing circuitry 80 of external device 12, processing circuitry 98 of server 94, and/or processing circuitry of one or more computing devices 100. In some examples, processing circuitry may obtain one or more EGMs, that was measured within a baseline period of time, such as within 30-days of implant of IMD 10, and determine a baseline EGM for a respective patient based on the EGMs obtained during the baseline period of time. In some examples, the baseline period of time may be 5-days, 10-days, 15-days, 20-days, 25-days. In some examples, the baseline period of time may be a number of days not explicitly listed, such as number of days less than, between, or greater than the various periods of times listed for the baseline period of time.

Processing circuitry 50 may apply obtained baseline EGM(s) and obtained follow-up EGM(s) to a machine learning model to determine an HF probability for each of the baseline EGM and the follow-up EGM, such as a baseline HF probability and a follow-up HF probability. In some examples, HF probability may correspond to an HF risk of the patient. Processing circuitry 50 may determine an amount of change between baseline HF probability and follow-up HF probability. Processing circuitry 50 may compare the determined amount of change to an HF hospitalization threshold to determine a risk of HF related hospitalization over an upcoming period of time, such as within the next 90 days after the follow-up EGM was measured.

In some examples, HF probability may indicate an amount of likely of a patient experiencing HF within a particular period of time. In some examples, HF probability may be a raw number, such as an amount of HF probability. In some examples, an amount of HF probability may be a percentage chance of a patient experiencing HF within a particular period of time. As an example, the particular period of time may be 90-days. In other examples, the particular period of time may be 30-days, 60-days, or 120-days. In some examples, the particular period of time may be a number of days not explicitly listed, such as number of days less than, between, or greater than the various periods of times listed for the particular period of time. An example HF probability may be a 5% chance of experiencing HF within a period of time. Other example HF probabilities may be any number greater than or equal to 0% and less than or equal to 100%. In some examples, an HF probability may be represented by other numbers other than percentages. In some examples, processing circuitry 50 may analyze a cardiac EGM sensed by IMD 10 to determine a particular HF probability based on converting a respective cardiac EGM signal to a time-frequency domain using a continuous wavelet transform and applying the converted EGM signal to a convolutional neural network to determine an HF probability. In some examples, processing circuitry 50 may determine a raw number of the HF probability using a regression model.

In some examples, processing circuitry 50 may determine a classification of ejection fraction based on the HF probability. For example, processing circuitry 50 may determine a baseline ejection fraction based on the baseline HF probability and/or may determine a follow-up ejection fraction based on the follow-up HF probability.

For example, processing circuitry 50 may apply single lead EGMs sensed by IMD 10 for respective time periods to a machine learning model, which may output respective values related to a probability of reduced EF for the time periods and, consequently, related to HF hospitalization risk for the time periods. The processing circuitry 50 may compare a change in probability over time periods, e.g., between a current time period and a baseline time period, to one or more thresholds, and generate communications or alerts to users and computing devices, or take one or more other actions based on the comparisons, e.g., based on the change in probability exceeding a threshold change indicative of increased risk of HF hospitalization over the future time period.

For example, an HF hospitalization risk threshold may be 0.1 (e.g., 10%) amount of change between a baseline HF probability and a follow-up HF probability. For example, in response to determining a change between a baseline HF probability and a follow-up HF probability is less than an HF hospitalization risk threshold, such as 0.1, processing circuitry 50 may determine patient has a “low risk” of having a HF hospitalization over a period of time, such as 90-days. In some examples, in response to determining a change between a baseline HF probability and a follow-up HF probability is greater than or equal to an HF hospitalization risk threshold, such as 0.1, processing circuitry 50 may determine patient has a “high risk” of having a HF hospitalization over a period of time after the follow-up EGM was measured, such as 90-days. In some examples the period of time after the follow-up EGM was measured may be 30-days, 60-days, or 120-days. In some examples, the period of time after the follow-up EGM was measured may be a number of days not explicitly listed, such as number of days less than, between, or greater than the various periods of times listed for the period of time after the follow-up EGM was measured.

In some examples, processing circuitry 50 may generate an output to indicate a particular patient as having a “low risk” or “high risk” of HF hospitalization over the period of time after the follow-up EGM was measured.

In order to determine the HF hospitalization risk threshold and/or to train the machine learning model(s) described herein, processing circuitry of system 2, e.g., processing circuitry 98 of server 94, may identify one or more patients having LVEF measurements obtained during a time period after IMD 10 was implanted, such as 30-days, 60-days, 90-days, 120-days, 365-days, or other number of days less than, between, or greater than the various periods of times listed. Processing circuitry 50 may determine a whether patient has an LVEF less than an LVEF threshold after implant of IMD 10. For example, after 60-days of implant of IMD 10. Processing circuitry 50 may obtain one or more follow-up EGMs. In some examples, processing circuitry 50 may obtain one or more follow-up EGMs from a patient determined to have LVEF less than an LVEF threshold. Processing circuitry 50 may obtain a follow-up EGM that is measured after an observation period of time after the baseline EGM was measured. In some examples, an observation period of time may be 60-days, 90-days, or 120-days. In some examples, the observation period of time may be a number of days not explicitly listed, such as number of days less than, between, or greater than the various periods of times listed for the observation period of time.

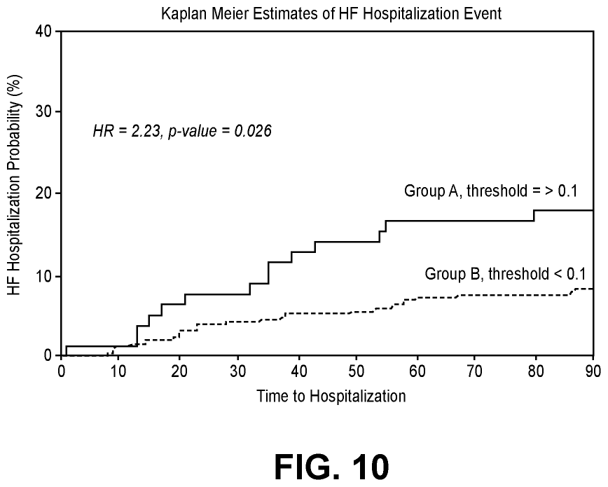

FIG. 10 is a graph illustrating example Kaplan Meier curves of HF hospitalization event estimates. As an example, as shown in FIG. 10, a total of 453 non overlapping follow-up EGMs/LVEFs (>90 days between observations) were analyzed from 367 unique patients. The patients in Group A had a change between a baseline HF probability and a follow-up HF probability greater than an HF hospitalization threshold of 0.1. The patients in Group B had a change between a baseline HF probability and a follow-up HF probability less than an HF hospitalization threshold of 0.1. There were 78 observations in Group A where about 18% had an HF hospitalization event and 375 observations in Group B where about 8% had an HF hospitalization event. The Kaplan Meier curve, as shown in FIG. 10, indicated the HF hospitalization event probability of Group A at day 90 was 17.9 and the HF hospitalization event probability of Group B at day 90 was 8.5. The HF hospitalization event probability of Group A at day 90 was higher than the HF hospitalization event probability of Group B at day 90.

In accordance with techniques of this disclosure, an increased HF hospitalization risk may be detected before a patient needs to go a hospital. Accordingly, medical intervention and/or treatment due to an increased HF hospitalization risk may be applied sooner which may lower the risk of long-term complications of heart failure patients and may reduce mortality and/or morbidity.

Although described in the context of examples in which a determination of risk or increased risk of HF hospitalization is based solely on application of single lead EGMs over time to a machine learning model, in other examples processing circuitry may determine the risk of HF hospitalization based on one or more additional physiological parameters. For example, the processing circuitry may utilize physiological parameters and techniques described in commonly-assigned U.S. application Ser. Nos. 12/184,149 and 12/184,003 by Sarkar et al., entitled “USING MULTIPLE DIAGNOSTIC PARAMETERS FOR PREDICTING HEART FAILURE EVENTS,” and “DETECTING WORSENING HEART FAILURE BASED ON IMPEDANCE MEASUREMENTS,” both filed on Jul. 31, 2008, both of which are incorporated herein by reference in their entirety. In some examples, the processing circuitry may compare the change in probability relative to the baseline to one or more thresholds to determine one or more evidence levels of HF hospitalization risk according to the techniques of U.S. application Ser. Nos. 12/184,149 and 12/184,003.

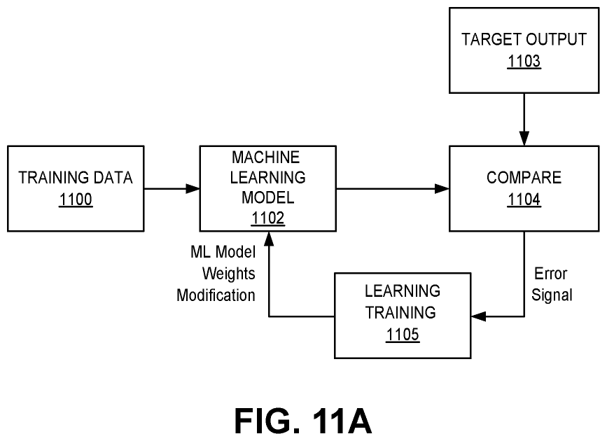

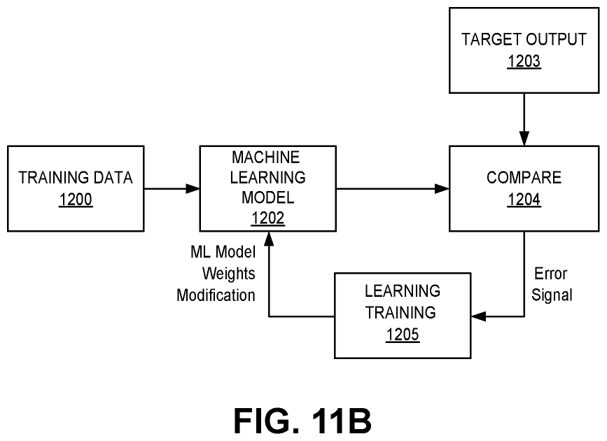

FIG. 11A is an example of a machine learning model 1102 being trained using supervised and/or reinforcement learning techniques. Machine learning model 1102 may correspond to any machine learning model described herein, e.g., machine learning model 810 illustrated in FIG. 8A or 8B. The machine learning model 1102 may be implemented using any number of models for supervised and/or reinforcement learning, such as but not limited to, an artificial neural network, a decision tree, naïve Bayes network, support vector machine, or k-nearest neighbor model, to name only a few examples. In some examples, one or more of IMD 10, external device 12, server 94, and/or computing device(s) 100 initially trains the machine learning model 1102 based on a training set of metrics and corresponding to an amount of ejection fraction and/or classification of ejection fraction. The training set 1100 may include a set of feature vectors, where each feature in the feature vector represents a value for a particular metric. One or more of IMD 10, external device 12, server 94, and/or computing device(s) 100 may select a training set comprising a set of training instances, each training instance comprising an association between one or more respective cardiac electrograms, EGM signals converted to a time-frequency domain using a continuous wavelet transform, and/or image representations of EGM signals converted to a time-frequency domain and an amount of ejection fraction and/or a classification of ejection fraction. A prediction or classification by the machine learning model 1102 may be compared 1104 to the target output 1103, and an error signal and/or machine learning model weights modification may sent/applied to the machine learning model 1102 based on the comparison to learn/train 1105 the machine learning model to modify/update the machine learning model 1102. For example, one or more of IMD 10, external device 12, server 94, and/or computing device(s) 100 may, for each training instance in the training set, modify, based on the respective cardiac electrogram, EGM signals converted to a time-frequency domain using a continuous wavelet transform, image representations of EGM signals converted to a time-frequency domain, and/or the amount of ejection fraction and/or classification of ejection fraction of the training instance, the machine learning model 1102 to change a score generated by the machine learning model 1102 in response to subsequent cardiac electrograms, respective EGM signals converted to a time-frequency domain using a continuous wavelet transform, and/or image representations of EGM signals converted to a time-frequency domain applied to the machine learning model 1102.

FIG. 11B is an example of a machine learning model 1202 being trained using supervised and/or reinforcement learning techniques. Machine learning model 1202 may correspond to any machine learning model described herein, e.g., machine learning model 810 illustrated in FIG. 8A or 8B. The machine learning model 1202 may be implemented using any number of models for supervised and/or reinforcement learning, such as but not limited to, an artificial neural network, a decision tree, naïve Bayes network, support vector machine, or k-nearest neighbor model, to name only a few examples. In some examples, one or more of IMD 10, external device 12, server 94, and/or computing device(s) 100 initially trains the machine learning model 1202 based on a training set of metrics and corresponding to a risk of hospitalization. The training set 1200 may include a set of feature vectors, where each feature in the feature vector represents a value for a particular metric. One or more of IMD 10, external device 12, server 94, and/or computing device(s) 100 may select a training set comprising a set of training instances, each training instance comprising an association between one or more cardiac electrograms and/or EGM signals converted to a time-frequency domain using a continuous wavelet transform, and a risk of hospitalization. A prediction or classification by the machine learning model 1202 may be compared 1204 to the target output 1203, and an error signal and/or machine learning model weights modification may sent/applied to the machine learning model 1202 based on the comparison to learn/train 1205 the machine learning model to modify/update the machine learning model 1202. For example, one or more of IMD 10, external device 12, server 94, and/or computing device(s) 100 may, for each training instance in the training set, modify, based on the respective cardiac electrogram, EGM signals converted to a time-frequency domain using a continuous wavelet transform, and/or a risk of hospitalization of the training instance, the machine learning model 1202 to change a score generated by the machine learning model 1202 in response to subsequent cardiac electrograms and/or EGM signals converted to a time-frequency domain using a continuous wavelet transform applied to the machine learning model 1202.

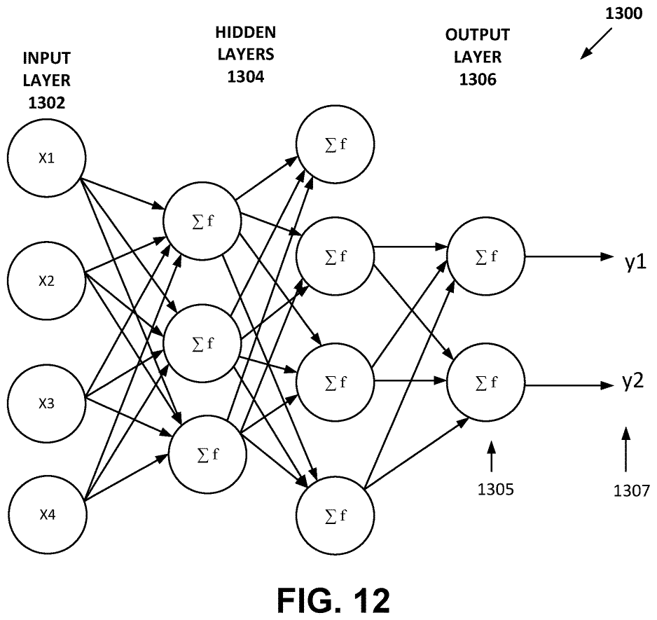

FIG. 12 is a conceptual diagram illustrating an example machine learning model 1300 configured to generate one or more values indicative of a risk of a health event, e.g., for heart failure or another patient condition, based on physiological parameter values, e.g., sensed by an IMD and/or other devices as described herein. Machine learning model 1300 is an example of a deep learning model, or deep learning algorithm. One or more of IMD 10, external device 12, or sever 94 may train, store, and/or utilize machine learning model 1300, but other devices may apply inputs associated with a particular patient to machine learning model 1300 in other examples. Some non-limiting examples of machine learning techniques include Bayesian probability models, Support Vector Machines, K-Nearest Neighbor algorithms, and Multi-layer Perceptron.

As shown in the example of FIG. 12, machine learning model 1400 may include three layers. These three layers include input layer 1302, hidden layer 1304, and output layer 1306. Output layer 1306 comprises the output from the transfer function 1305 of output layer 1306. Input layer 1302 represents each of the input values X1 through X4 provided to machine learning model 1300. The number of inputs may be less than or greater than 4, including much greater than 4, e.g., hundreds or thousands, In some examples, the input values may be any of the of physiological or other patient parameter values described herein. In some examples, the input values may include a time-frequency representation of EGM signal, such as a two-dimensional array or sequence, and/or image representation of time-frequency representation of EGM signal.

Each of the input values for each node in the input layer 1302 is provided to each node of hidden layer 1304. In the example of FIG. 12, hidden layers 1304 include two layers, one layer having four nodes and the other layer having three nodes, but fewer or greater number of nodes may be used in other examples. Each input from input layer 1302 is multiplied by a weight and then summed at each node of hidden layers 1304. During training of machine learning model 1300, the weights for each input are adjusted to establish the relationship between input values and one or more output values indicative of an amount of ejection fraction or a classification of ejection fraction. In some examples, one hidden layer may be incorporated into machine learning model 1300, or three or more hidden layers may be incorporated into machine learning model 1300, where each layer includes the same or different number of nodes.

The result of each node within hidden layers 1304 is applied to the transfer function of output layer 1306. The transfer function may be liner or non-linear, depending on the number of layers within machine learning model 1300. Example non-linear transfer functions may be a sigmoid function or a rectifier function. The output 1307 of the transfer function may be a value or values indicative of a risk of an HF event or other health event of the patient. By applying the time-frequency representation of EGM signal, such as a two-dimensional array or sequence, and/or image representation of time-frequency representation of EGM signal to a machine learning model, such as machine learning model 1300, processing circuitry of system 2 is able to determine the amount of ejection fraction or a classification of ejection fraction with great accuracy, specificity, and sensitivity.

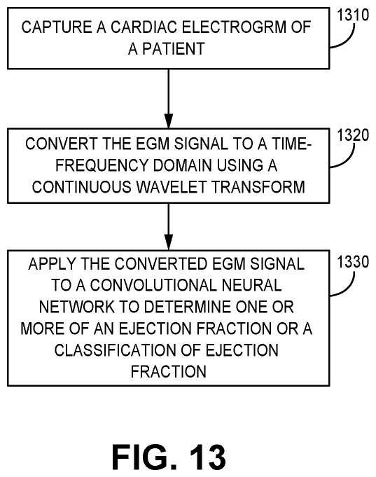

FIG. 13 is a flow diagram illustrating an example technique for medical system 2. As indicated by FIG. 13, two or more electrodes forming a signal lead capture an EGM signal of a patient (1310). Processing circuitry 50 may convert the EGM signal to a time-frequency domain using a continuous wavelet transform (1320). Processing circuitry 50 may apply the converted EGM signal to a convolutional neural network to determine one or more of an amount of ejection fraction or classification of ejection fraction (1330).

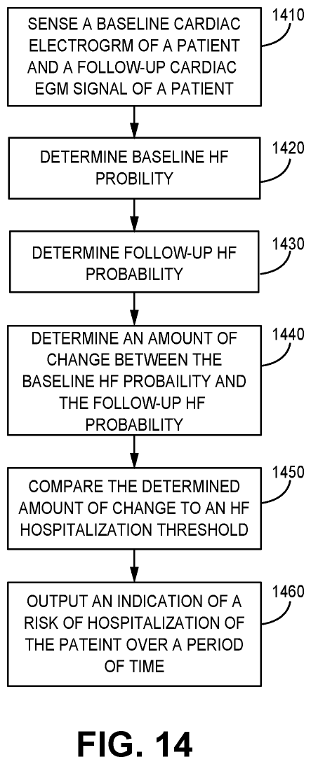

FIG. 14 is a flow diagram illustrating an example technique for medical system 2. As indicated by FIG. 14, two or more electrodes forming a signal lead sense a baseline EGM signal and a follow-up EGM signal of a patient (1410). Processing circuitry 50 may determine a baseline HF probability based on the baseline cardiac EGM signal (1420). Processing circuitry 50 may determine a follow-up HF probability based on the follow-up cardiac EGM signal (1430). Processing circuitry 50 may determine an amount of change between the baseline HF probability and the follow-up HF probability (1440). Processing circuitry 50 may compare the determined an amount of change to an HF hospitalization threshold (1450). Processing circuitry 50 may output an indication of a risk of hospitalization of the patient over a period of time is high in response to determining the amount of change between the baseline HF probability and the HF probability is greater than or equal to the HF hospitalization threshold (1460).

The techniques described in this disclosure may be implemented, at least in part, in hardware, software, firmware, or any combination thereof. For example, various aspects of the techniques may be implemented within one or more microprocessors, DSPs, ASICs, FPGAs, or any other equivalent integrated or discrete logic QRS circuitry, as well as any combinations of such components, embodied in external devices, such as physician or patient programmers, stimulators, or other devices. The terms “processor” and “processing circuitry” may generally refer to any of the foregoing logic circuitry, alone or in combination with other logic circuitry, or any other equivalent circuitry, and alone or in combination with other digital or analog circuitry.

For aspects implemented in software, at least some of the functionality ascribed to the systems and devices described in this disclosure may be embodied as instructions on a computer-readable storage medium such as RAM, DRAM, SRAM. magnetic discs, optical discs, flash memories, or forms of EPROM or EEPROM. The instructions may be executed to support one or more aspects of the functionality described in this disclosure.

In addition, in some aspects, the functionality described herein may be provided within dedicated hardware and/or software modules. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components. Also, the techniques could be fully implemented in one or more circuits or logic elements. The techniques of this disclosure may be implemented in a wide variety of devices or apparatuses, including an IMD, an external programmer, a combination of an IMD and external programmer, an integrated circuit (IC) or a set of ICs, and/or discrete electrical circuitry, residing in an IMD and/or external programmer.

The following examples are illustrative of the techniques described herein.
Example 1A: A medical system for determining ejection fraction includes two or more electrodes forming a single lead configured to capture a cardiac electrogram (EGM) signal of a patient; and circuitry configured to: convert the EGM signal to a time-frequency domain using a continuous wavelet transform; convert the converted EGM signal to one or more images; and apply the one or more images to a convolutional neural network to determine one or more of an amount of ejection fraction or a classification of ejection fraction.
Example 2A: The medical system of example 1A, wherein the convolutional neural network is one-dimensional.
Example 3A: The medical system of any of examples 1A-2A, wherein the ejection fraction is a ventricular ejection fraction.
Example 4A: The medical system of any of examples 1A-3A, wherein the circuitry is further configured to determine heart failure risk of the patient by comparing an amount of the determined ejection fraction to a threshold.
Example 5A: The medical system of any of examples 1A-4A, wherein the circuitry is further configured to determine the patient is at high risk of heart failure when the determined amount of ejection fraction is below a threshold.
Example 6A: The medical system of example 5A, wherein the threshold is 35%.
Example 7A: The medical system of any of examples 1A-6A, wherein the circuitry is further configured to: determine an amount of change in ejection fraction over a period of time; and determine heart failure risk of the patient based on the change in ejection fraction over the period of time.
Example 8A: The medical system of any of examples 1A-7A, wherein the circuitry is further configured to cause the determined one or more of an amount of ejection fraction or a classification of ejection fraction to be output to a clinician computing device to determine treatment or recommend additional testing based on the determined one or more of an amount of ejection fraction or a classification of ejection fraction.
Example 9A: The medical system of any of examples 1A-8A, wherein the one or more converted images comprise one or more scalograms.
Example 10A: A method for operating processing circuity of a medical system includes receiving, by the processing circuitry, a cardiac electrogram (EGM) signal of a patient obtained by a single lead; converting, by the processing circuitry, the EGM signal to a time-frequency domain using a continuous wavelet transform; converting, by the processing circuitry, the converted EGM signal to one or more images; and applying, by the processing circuitry, the converted one or more images to a convolutional neural network to determine one or more of an amount of ejection fraction or a classification of ejection fraction.
Example 11A: The method of example 10A, wherein the convolutional neural network is one-dimensional.
Example 12A: The method of any of examples 10A-11A, wherein the ejection fraction is a ventricular ejection fraction.
Example 13A: The method of any of examples 10A-12A, wherein the method further comprises determining, by the processing circuitry, a heart failure risk of the patient by comparing an amount of the determined ejection fraction to a threshold.
Example 14A: The method of any of examples 10A-13A, wherein the method further comprises determining, by the processing circuitry, the patient is at high risk of heart failure when the determined amount of ejection fraction is below a threshold.
Example 15A: The method of example 14A, wherein the threshold is 35%.
Example 16A: The method of any of examples 10A-15A, wherein the method further comprises: determining, by the processing circuitry, an amount of change in ejection fraction over a period of time; and determining, by the processing circuitry, heart failure risk of the patient based on the change in ejection fraction over the period of time.
Example 17A: The method of any of examples 10A-16A, wherein the method further comprises: outputting, by the processing circuitry, the determined one or more of an amount of ejection fraction or a classification of ejection fraction to a clinician computing device to determine treatment or recommend additional testing based on the determined one or more of an amount of ejection fraction or a classification of ejection fraction.
Example 18A: The method of any of examples 10A-17A, wherein the one or more converted images are scalograms.
Example 1: A medical system for determining ejection fraction includes two or more electrodes forming a single lead configured to capture a cardiac electrogram (EGM) signal of a patient; and circuitry configured to: convert the EGM signal to a time-frequency domain using a continuous wavelet transform; and apply the converted EGM signal to a convolutional neural network to determine one or more of an amount of ejection fraction or a classification of ejection fraction.
Example 2: The medical system of example 1, wherein the circuitry is further configured to: select components from the converted EGM signal based on relevance; and store the selected components to a two-dimensional array.
Example 3: The medical system of example 2, wherein the circuitry is further configured to apply the stored selected components to the convolutional neural network to determine one or more of an amount of ejection fraction or a classification of ejection fraction.
Example 4: The medical system of any of examples 1-3, wherein the convolutional neural network is one-dimensional.
Example 5: The medical system of any of examples 1-4, wherein the ejection fraction is a ventricular ejection fraction.
Example 6: The medical system of any of examples 1-5, wherein the circuitry is further configured to determine heart failure risk of the patient by comparing an amount of the determined ejection fraction to a threshold.
Example 7: The medical system of any of examples 1-6, wherein the circuitry is further configured to determine the patient is at high risk of heart failure when the determined amount of ejection fraction is below a threshold.
Example 8: The medical system of example 7, wherein the threshold is 35%.
Example 9: The medical system of any of examples 1-8, wherein the circuitry is further configured to: determine an amount of change in ejection fraction over a period of time; and determine heart failure risk of the patient based on the change in ejection fraction over the period of time.
Example 10: The medical system of any of examples 1-9, wherein the circuitry is further configured to cause the determined one or more of an amount of ejection fraction or a classification of ejection fraction to be output to a clinician computing device to determine treatment or recommend additional testing based on the determined one or more of an amount of ejection fraction or a classification of ejection fraction.
Example 11: A method for operating processing circuity of a medical system includes receiving, by the processing circuity, a cardiac electrogram (EGM) signal of a patient obtained by a single lead; converting, by the processing circuity, the EGM signal to a time-frequency domain using a continuous wavelet transform; and applying, by the processing circuity, the converted EGM signal to a convolutional neural network to determine one or more of an amount of ejection fraction or a classification of ejection fraction.
Example 12: The method of example 11, wherein the method further comprises: selecting, by the processing circuity, components from the converted EGM signal based on relevance; and storing, by the processing circuity, the selected components to a two-dimensional array.
Example 13: The method of example 12, wherein the method further comprises: applying, by the processing circuity, the stored selected components to the convolutional neural network to determine one or more of an amount of ejection fraction or a classification of ejection fraction.
Example 14: The method of any of examples 11-13, wherein the convolutional neural network is one-dimensional.
Example 15: The method of any of examples 11-14, wherein the ejection fraction is a ventricular ejection fraction.
Example 16: The method of any of examples 11-15, wherein the method further comprises determining, by the processing circuity, a heart failure risk of the patient by comparing an amount of the determined ejection fraction to a threshold.
Example 17: The method of any of examples 11-16, wherein the method further comprises determining, by the processing circuity, the patient is at high risk of heart failure when the determined amount of ejection fraction is below a threshold.
Example 18: The method of example 17, wherein the threshold is 35%.
Example 19: The method of any of examples 11-18, wherein the method further comprises: determining, by the processing circuity, an amount of change in ejection fraction over a period of time; and determining, by the processing circuity, heart failure risk of the patient based on the change in ejection fraction over the period of time.
Example 20: The method of any of examples 10-19, wherein the method further comprises: outputting, by the processing circuity, the determined one or more of an amount of ejection fraction or a classification of ejection fraction to a clinician computing device to determine treatment or recommend additional testing based on the determined one or more of an amount of ejection fraction or a classification of ejection fraction.
Example 21: A medical system for determining heart failure risk includes two or more electrodes forming a single lead configured to sense a baseline cardiac electrogram (EGM) signal of a patient and a follow-up cardiac EGM signal of the patient; and circuitry configured to: determine a baseline heart failure (HF) probability based on the baseline cardiac EGM signal; determine a follow-up HF probability based on the follow-up cardiac EGM signal; determine an amount of change between the baseline HF probability and the follow-up HF probability; compare the determined amount of change to an HF hospitalization threshold; and in response to determining the amount of change between the baseline HF probability and the follow-up HF probability is greater than or equal to the HF hospitalization threshold, output an indication of a risk of hospitalization of the patient over a period of time is high.
Example 22: The medical system of example 21, wherein the period of time is 90 days.
Example 23: The medical system of any of examples 21-22, wherein the HF hospitalization threshold is 10%.
Example 24: The medical system of any of examples 21-22, wherein the HF hospitalization threshold is 0.1.
Example 25; The medical system of any of examples 21-24, wherein the follow-up cardiac EGM signal is captured at least 60 days after the baseline cardiac EGM signal is captured.
Example 26: The medical system of any of examples 21-25, wherein the circuitry is further configured to output the indication of a risk of hospitalization to a clinician computing device to determine treatment or recommend additional testing based on the determined amount of change between the baseline HF probability and the follow-up HF probability.
Example 27: The medical system of any of examples 21-26, wherein the baseline HF probability indicates an amount ejection fraction during a baseline period of time and the follow-up HF probability indicates an amount ejection fraction after an observation period of time.

Various examples have been described. These and other examples are within the scope of the following claims.

1. A medical system for determining ejection fraction comprising:
two or more electrodes forming a single lead configured to capture a cardiac electrogram (EGM) signal of a patient; and
circuitry configured to:
convert the EGM signal to a time-frequency domain using a continuous wavelet transform;
convert the converted EGM signal to one or more images; and
apply the one or more images to a convolutional neural network to determine one or more of an amount of ejection fraction or a classification of ejection fraction.

2. The medical system of claim 1, wherein the convolutional neural network is one-dimensional.

3. The medical system of claim 1, wherein the ejection fraction is a ventricular ejection fraction.

4. The medical system of claim 1, wherein the circuitry is further configured to determine heart failure risk of the patient by comparing an amount of the determined ejection fraction to a threshold.

5. The medical system of claim 1, wherein the circuitry is further configured to determine the patient is at high risk of heart failure when the determined amount of ejection fraction is below a threshold.

6. The medical system of claim 5, wherein the threshold is 35%.

7. The medical system of claim 1, wherein the circuitry is further configured to:
determine an amount of change in ejection fraction over a period of time; and
determine heart failure risk of the patient based on the change in ejection fraction over the period of time.

8. The medical system of claim 1, wherein the circuitry is further configured to cause the determined one or more of an amount of ejection fraction or a classification of ejection fraction to be output to a clinician computing device to determine treatment or recommend additional testing based on the determined one or more of an amount of ejection fraction or a classification of ejection fraction.

9. The medical system of claim 1, wherein the one or more converted images comprise one or more scalograms.

10. A method for operating processing circuity of a medical system comprising:
receiving, by the processing circuitry, a cardiac electrogram (EGM) signal of a patient obtained by a single lead;
converting, by the processing circuitry, the EGM signal to a time-frequency domain using a continuous wavelet transform;
converting, by the processing circuitry, the converted EGM signal to one or more images; and
applying, by the processing circuitry, the converted one or more images to a convolutional neural network to determine one or more of an amount of ejection fraction or a classification of ejection fraction.

11. The method of claim 10, wherein the convolutional neural network is one-dimensional.

12. The method of claim 10, wherein the ejection fraction is a ventricular ejection fraction.

13. The method of claim 10, wherein the method further comprises:
determining, by the processing circuitry, a heart failure risk of the patient by comparing an amount of the determined ejection fraction to a threshold.

14. The method of claim 10, wherein the method further comprises:
determining, by the processing circuitry, the patient is at high risk of heart failure when the determined amount of ejection fraction is below a threshold.

15. The method of claim 14, wherein the threshold is 35%.

16. The method of claim 10, wherein the method further comprises:
determining, by the processing circuitry, an amount of change in ejection fraction over a period of time; and determining, by the processing circuitry, heart failure risk of the patient based on the change in ejection fraction over the period of time.

17. The method of claim 10, wherein the method further comprises:
outputting, by the processing circuitry, the determined one or more of an amount of ejection fraction or a classification of ejection fraction to a clinician computing device to determine treatment or recommend additional testing based on the determined one or more of an amount of ejection fraction or a classification of ejection fraction.

18. The method of claim 10, wherein the one or more converted images are scalograms.

19. A computer-readable medium comprising instructions that, when executed, causes processing circuitry of a medical system to:
receive a cardiac electrogram (EGM) signal of a patient obtained by a single lead;
convert the EGM signal to a time-frequency domain using a continuous wavelet transform;
convert the converted EGM signal to one or more images; and
apply the one or more images to a convolutional neural network to determine one or more of an amount of ejection fraction or a classification of ejection fraction.
